(19) United States
(12) Patent Application Publication
(10) Pub. No.: US 2012/0116459 A1
(43) Pub. Date: May 10, 2012

(54) Bone Fixation Device and Methods for Use Thereof

(76) Inventor: Eric W. Nottmeier, Atlantic Beach, FL (US)

(21) Appl. No.: 13/285,503

(22) Filed: Oct. 31, 2011

Related U.S. Application Data
(60) Provisional application No. 61/409,426, filed on Nov. 2, 2010.

(51) Int. Cl. A61B 17/86; A61B 17/88

(52) U.S. Cl. 606/279; 606/304

(57) ABSTRACT

A bone fixation device for fixing a first bone to a second bone includes a self tapping screw having an aperture extending along a longitudinal axis of the screw, and a driver having an aperture extending from a proximal end to a distal end along a longitudinal axis of the driver, the distal end being configured to engage the screw such that the aperture of the driver and the aperture of the screw are coaxially aligned, thereby defining a lumen. A guide rod having a tapered tip for piercing bone tissue is shaped and dimensioned to extend through the lumen and configured to fasten to the driver. A blunt-ended rod advances a bioactive agent through the lumen, the blunt-ended rod being dimensioned to extend through the lumen to decrease the injury to vital structures as the screw is advanced into the second bone.

### CROSS-REFERENCES TO RELATED APPLICATIONS

This application claims priority from U.S. Patent Application No. 61/409,426 filed Nov. 2, 2010.

### STATEMENT REGARDING FEDERALLY SPONSORED RESEARCH

Not Applicable.

### BACKGROUND OF THE INVENTION

1. Field of the Invention

The field of the invention is medical devices and methods for their use. More particularly, the invention relates to medical devices for bone fixation and arthrodesis and methods for their use.

2. Description of the Related Art

Transarticular facet screw fixation is often used for treating instability between vertebrae, such as the atlas (C1 vertebra) and axis (C2 vertebra). This technique can also be used to fixate vertebrae inferior to the C2 vertebra and provides rigid internal fixation of the unstable vertebrae. In a typical C1-C2 fixation procedure, a midline incision is made on a patient from the occiput to the spinous process of, for example, the C5 vertebra, thereby exposing the C1 and C2 vertebrae, among others. Depending on the alignment of the C1 and C2 vertebrae, the fixation procedure can be performed in a human H directly through the incision or, as illustrated in FIG. 1, percutaneously along alignment line A.

In a typical C1-C2 fixation procedure, a Kirschner wire (“K-wire”) is used to pierce the inferior facet of the C2 vertebra, run through the C2 vertebra, and pierce into the lateral mass of the C1 vertebra. Precise placement of the K-wire is necessary, or nearby vital structures, such as the vertebral artery and spinal nerves, can be damaged, resulting in injury to the patient. A cannulated drill is placed over the K-wire and used to drill a pilot hole, which is tapped, in the C1 and C2 vertebra. A cannulated screw is then placed over the K-wire and advanced through the C1 and C2 vertebra, guided by the pilot hole and the K-wire. After the screw is in place, the K-wire is removed. See, for example, Apostolides et al., “Technique of Posterior Atlantoaxial Arthrodesis With Transarticular Facet Screw Fixation and Interspinous Wiring”, Operative Techniques in Neurosurgery, Vol. 1, No. 2 (June), pp. 67-71, 1998.

Bone growth agents are typically administered during a bone fixation procedure to fuse the unstable bones. In some instances, a bone growth agent is administered through transverse openings in a cannulated bone fixation screw, so that the vertebrae being fixated can ultimately fuse together. See, for example, U.S. Pat. No. 7,354,442.

To assist the surgeon in efficiently, safely, and effectively performing a bone fixation procedure, it would be desirable to provide a single device that performs the tasks of multiple individual tools, prevents the damage of vital structures, and administers a bone growth agent in a manner conducive to promoting the fixation of the unstable bones.

### SUMMARY OF THE INVENTION

The present invention overcomes the aforementioned drawbacks by providing a bone fixation device that utilizes a self tapping screw and sharp guide rod that is interchangeable with a blunt-ended rod that is configured to prevent damage to vital structures. Such a device includes a self tapping screw having an aperture extending along a longitudinal axis of the screw, and a driver having an aperture extending from a proximal end to a distal end along a longitudinal axis of the driver, the distal end being configured to engage the screw such that the aperture of the driver and the aperture of the screw are coaxially aligned, thereby defining a lumen. Also included is a guide rod having a tapered tip for piercing bone tissue, the guide rod being shaped and dimensioned to extend through the lumen and configured to fasten to the driver, and a blunt-ended rod for advancing a bioactive agent through the lumen, the blunt-ended rod also being shaped and dimensioned to extend through the lumen.

It is an aspect of the invention to provide a method for fixing a first bone to a second bone, such as two cervical or other vertebrae using a bone fixation device. Such a method includes the steps of providing the device to a surgeon, the device generally including a cannulated self tapping screw, a cannulated driver configured to coaxially engage the cannulated screw; a guide rod shaped and dimensioned to engage the cannulated screw and cannulated driver, and having a tapered end for piercing a bone tissue; and a blunt-ended rod shaped and dimensioned to engage the cannulated screw and cannulated driver, and having a blunt end for advancing a bioactive agent through a lumen formed when the cannulated driver engages the cannulated screw. The cannulated driver engages the cannulated screw, thereby defining a lumen running through both the screw and the driver. The guide rod is advanced through the lumen so that the guide rod pierces a surface of the first bone, and the guide rod is fastened to the cannulated driver. The cannulated driver is operated to collectively advance the cannulated screw and the guide rod through the first bone until the guide rod pierces a surface of the second bone, forming a pilot hole in that bone. The guide rod is then removed from the lumen of the device and a bioactive agent is provided to the lumen. Then, the blunt-ended rod is advanced through the lumen of the device, thereby dispersing the bioactive agent into a joint space between the first and second bones. The blunt-ended rod is then fastened to the cannulated driver and the cannulated driver is operated to collectively advance the cannulated screw and blunt-ended rod through the second bone, thereby fixing the first and second bones together. The cannulated driver and blunt-ended rod are then disengaged from the cannulated screw. The device can also be used to fixate fractures within a single bone.

The foregoing and other aspects and advantages of the invention will appear from the following description. In the description, reference is made to the accompanying drawings which form a part hereof, and in which there is shown by way of illustration an example embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 illustrates an exemplary surgical trajectory used during the percutaneous fixation of a C1 and C2 vertebra;

FIG. 2 is an elevation view of an exemplary bone fixation device in accordance with embodiments of the present invention;

FIG. 2A is a cross-sectional view of the exemplary bone fixation device of FIG. 2 viewed along line 2A-2A of FIG. 2;

FIG. 3 is an elevation view of an exemplary cannulated screw, cannulated driver, and guide rod that form a part of the exemplary bone fixation device of FIG. 2;

FIG. 4 is a perspective view showing the engagement of a distal end of the cannulated driver and the head of the cannulated driver of FIG. 2;

FIG. 5 is a pictorial view of the bone fixation device of FIG. 2 with a guide rod being operated to advance a screw through a first bone;

FIG. 5A is a partial cross-sectional view of FIG. 5 viewed along line 5A-5A of FIG. 5;

FIG. 6 is a pictorial view of the bone fixation device of FIG. 2 with a blunt-ended rod being operated to advance a bioactive agent through the lumen of the device and out the distal aspect of the screw into a joint space between the first bone and a second bone;

FIG. 6A is a partial cross-sectional view of FIG. 6 viewed along line 6A-6A of FIG. 6;

FIG. 7 is a pictorial view of the bone fixation device of FIG. 2 with a blunt-ended rod being operated to advance a screw through a second bone;

FIG. 7A is a partial cross-sectional view of FIG. 7 viewed along line 7A-7A of FIG. 7; and

FIG. 8 is a pictorial representation of the fixation of a C1 and C2 vertebra with a cannulated screw.

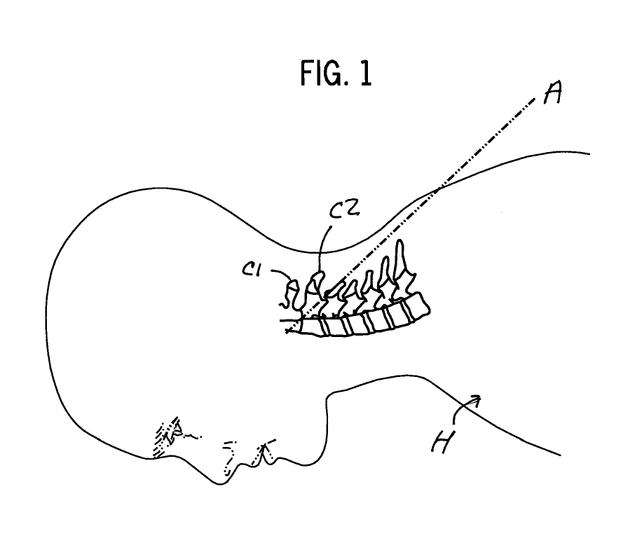

Like reference numerals will be used to refer to like parts from Figure to Figure in the following description of the drawings.

### DETAILED DESCRIPTION OF THE INVENTION

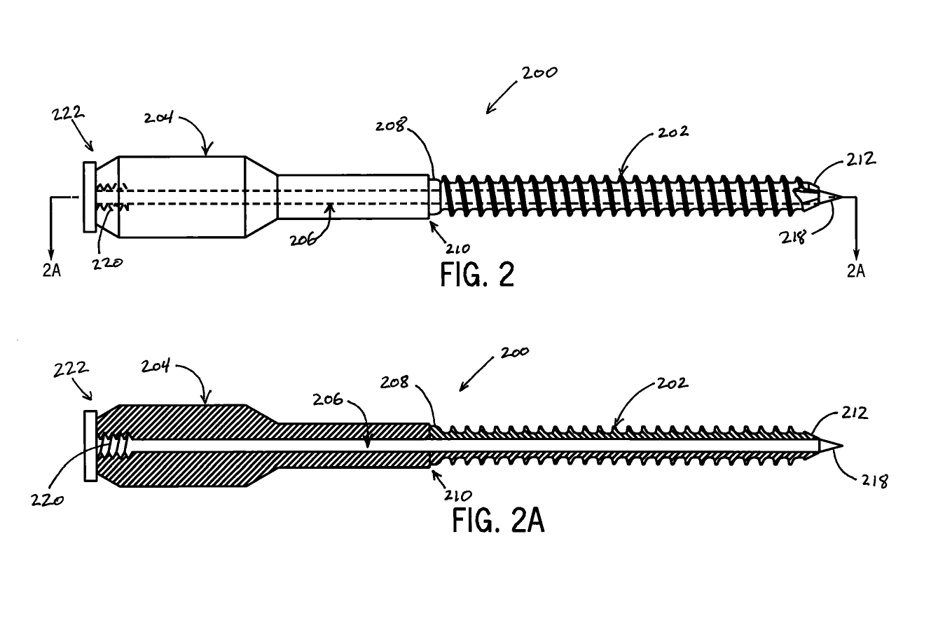

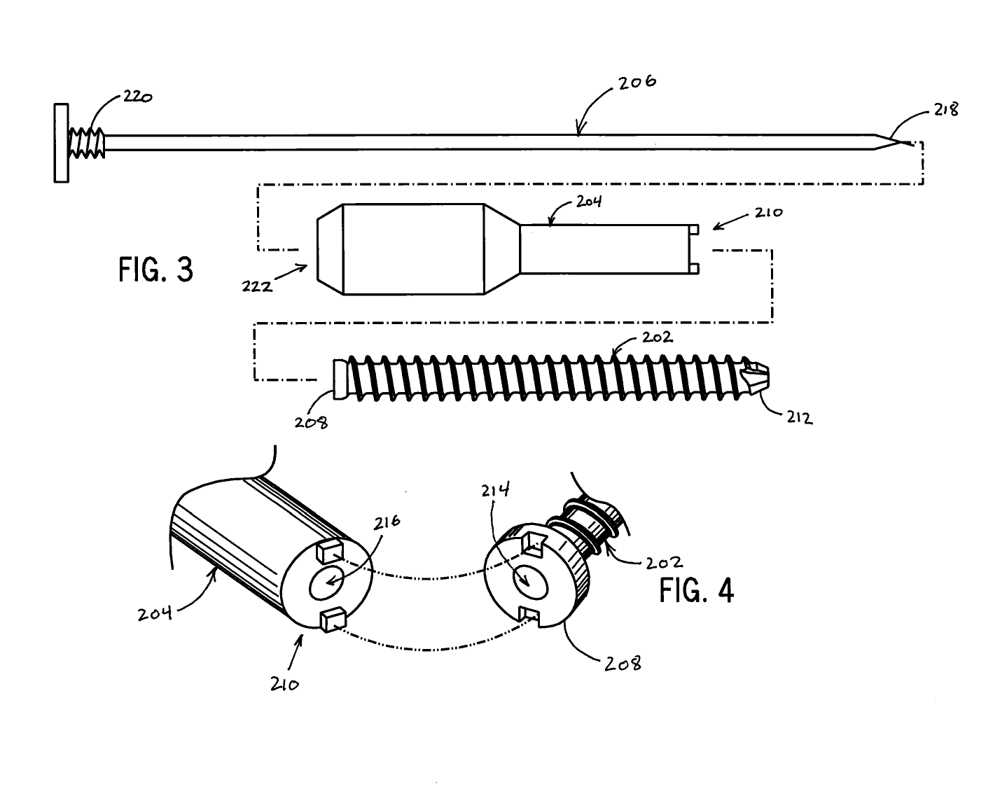

FIGS. 2, 2A, and 3 illustrate an exemplary bone fixation device 200 in accordance with the present invention. The bone fixation device 200 generally includes a cannulated screw 202, a cannulated driver 204, a guide rod 206, and a blunt-ended rod (not shown in FIGS. 2, 2A, and 3). As used herein, the term “cannulated” refers to an object having a hollow shaft, aperture, or lumen, running through it. Thus, a “cannulated screw” includes a screw having a hollow shaft, aperture, or lumen, running, for example, through the length of the screw. The cannulated screw 202, cannulated driver 204, guide rod 206, and blunt-ended rod are preferably composed of a material suitable for surgical use, such as stainless steel or titanium.

The head 208 of the cannulated screw 202 is configured to engage the distal end 210 of the cannulated driver 204, as illustrated in FIG. 4. The head 208 of the cannulated screw 202 may include any number of suitable drive types that allow for a centrally positioned lumen to run therethrough while still providing a portion that can be engaged by the distal end 210 of the cannulated driver 204. When the tip of the guide rod 218 is positioned through the axial distal tip 212 of the cannulated screw 202, the resulting configurations is for a self-drilling screw, so that it can be advanced through bone tissue without the need for a pilot hole to be drilled first. A self-tapping screw is a screw that can advance through a pilot hole without the need to tap the pilot hole first.

As illustrated in FIG. 4, the cannulated screw 202 has a lumen 214 running therethrough that is substantially coaxial with a lumen 216 running through the cannulated driver 204. As illustrated in FIGS. 2 and 2A, when the cannulated driver 204 engages the cannulated screw 202, a single lumen configured to receive the guide rod 206 is formed by the alignment of the lumens 214, 216. This single lumen is similarly configured to receive a blunt-ended rod, as will be described below.

The guide rod 206 has a sharp, tapered tip 218 at its distal end that is configured to pierce bone tissue. The proximal end of the guide rod 206 includes a locking mechanism 220 that is configured to securely engage the proximal end 222 of the cannulated driver 204 so that when the cannulated driver 204 is operated, the guide rod 206 moves in unison with the cannulated driver 204. For example, the locking mechanism 220 may include a series of threads disposed on a proximal portion of the shaft of the guide rod 206, and a series of similarly cut threads in the proximal portion of the lumen 216 in the cannulated driver 204 thereby provides the secure engagement of the guide rod 206 with the cannulated driver 204.

To prevent inadvertent damage to vital structures, such as the vertebral artery, the guide rod 206 is fastened to the cannulated driver 204, thereby effectively forming the tip of the cannulated screw 202 when it is engaged by the cannulated driver 204. Because the guide rod 206 is securely fastened to the cannulated driver 204, the guide rod 206 remains in a stationary position relative to the cannulated screw 202 during advancement of the cannulated screw 202. As the cannulated screw is advanced near vital structures, such as the vertebral artery, the guide rod 206 can be retracted and replaced with a blunt-ended rod. As will be described below, this blunt-ended rod may also serve the purpose of advancing a bioactive agent through the single lumen formed by lumens 214, 216. The blunt-ended rod is similar in construction to that of the guide rod 206, except that the tip of the blunt-ended rod is blunt so that it cannot pierce bone tissue or vital structures. Alternatively, the guide rod 206 can also have a blunt end as in rod 232 instead of the sharp end 218 shown in FIG. 3. Otherwise, the blunt-ended rod is composed of a similar material and includes a similarly configured locking mechanism.

Having generally described the features of the bone fixation device 200, a discussion of its general operation is now provided. The bone fixation device 200 is provided to a surgeon, who engages the cannulated screw 202 with the cannulated driver 204, thereby forming a single lumen by coaxially aligning the lumens 214, 216 of the cannulated screw 202 and cannulated driver 204. The guide rod 206 is then passed through the single lumen and secured to the cannulated driver 204 by way of the locking mechanism 220. For example, the guide rod 206 is threaded into a threaded portion of the cannulated driver 204 lumen 216.

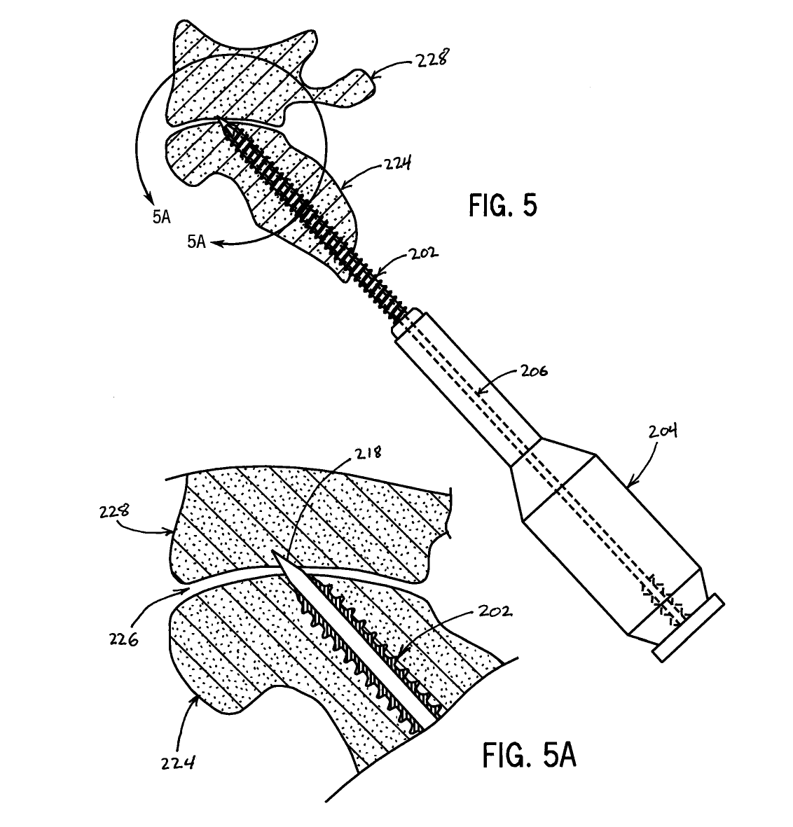

Referring now to FIGS. 5 and 5A, the assembled device 200 is positioned over the surface of a first bone 224 so that the exposed tip 218 of the guide rod 206 pierces the surface of the first bone 224. The cannulated driver 204 is then operated so as to advance the cannulated screw 202 through the first bone 224. Because the guide rod 206 is securely fastened to the cannulated driver 204, the cannulated screw 202 and the guide rod 206 are collectively advanced through the bone tissue. As illustrated in FIGS. 5 and 5A, the cannulated driver 204 is operated until the tip 218 of the guide rod 206 pierces through the first bone 224, crosses a joint space 226 between the first bone 224 and an adjacent second bone 228, and pierces the surface of the second bone 228. At this point, the guide rod 206 is removed from the lumen of the bone fixation device 200.

After the guide rod 206 has been removed from the bone fixation device 200, a bioactive agent 230 is provided to the lumen of the device. A “bioactive agent” as used herein includes, without limitation, physiologically or pharmacologically active substances that act locally or systemically in the body. A bioactive agent is a substance used for the treatment, prevention, diagnosis, cure or mitigation of disease or illness, or a substance which affects the structure or function of the body or which becomes biologically active or more active after it has been placed in a predetermined physiological environment. Bioactive agents include, without limitation, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, extracellular matrix and/or its individual components, pharmaceuticals, allograft bone, and therapeutics. Exemplary bioactive agents for use with the present invention include bone morphogenetic proteins (“BMPs”) and demineralized bone matrix (“DBM”) as these agents promote the growth of bone, thereby aiding the fixation process.

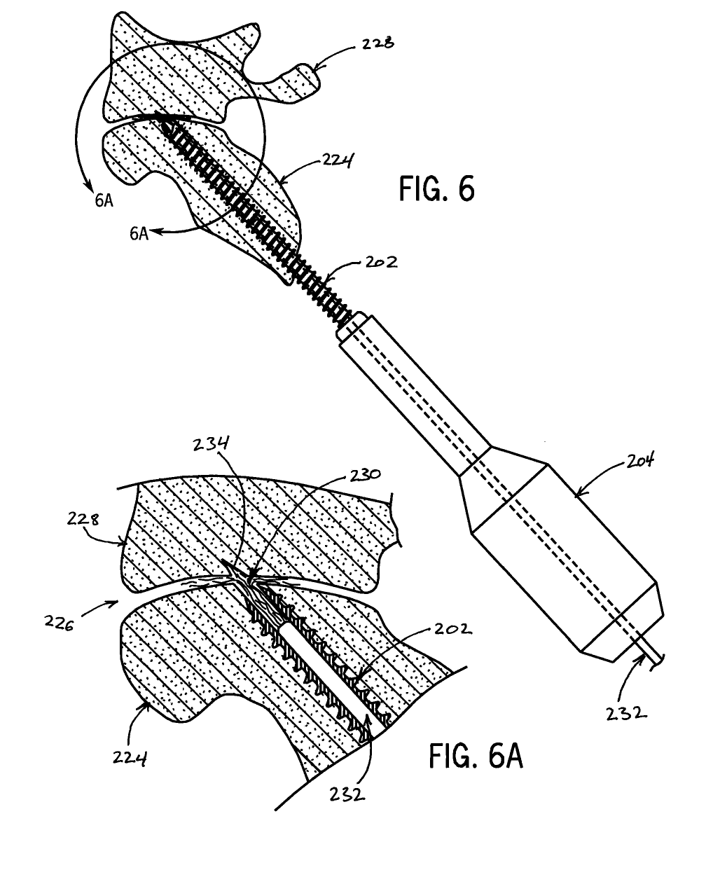

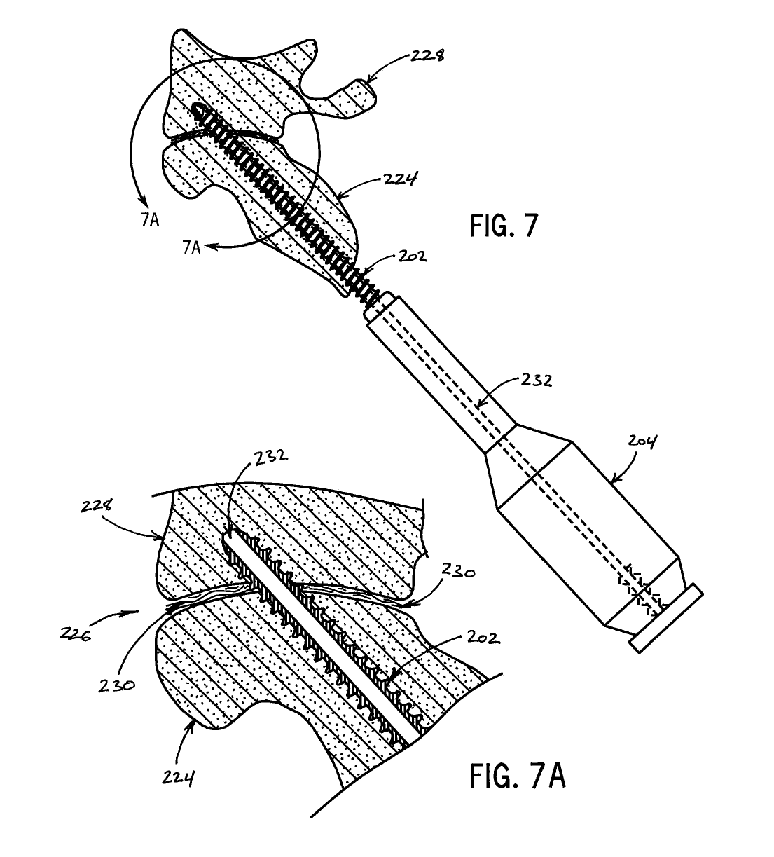

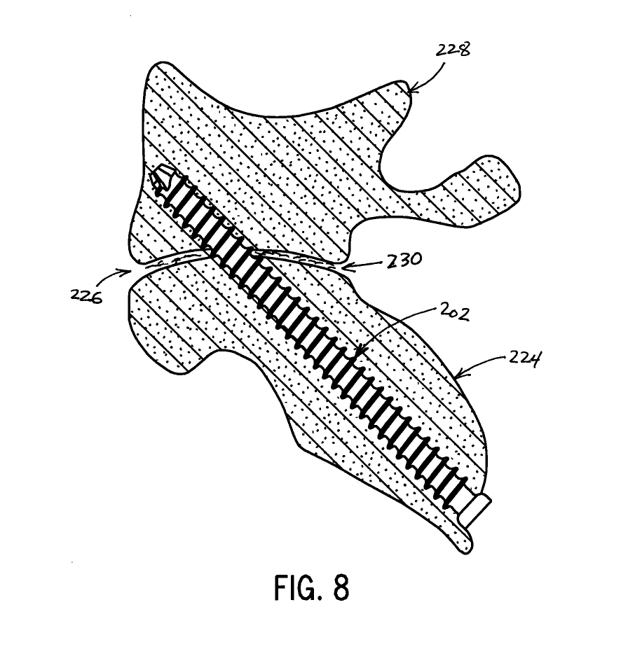

As illustrated in FIGS. 6 and 6A, after the bioactive agent 230 is provided to the lumen of the device 200, the blunt-ended rod 232 is advanced through the lumen of the device 200 so that the bioactive agent 230 is dispersed out of the tip 212 of the cannulated screw 202 and into the joint space 226. Because the guide rod 206 was allowed to pierce the surface of the second bone 228, a pilot hole 234 is provided in the second bone 228 for the advancement of the cannulated screw 202 into the bone tissue. Because the cannulated screw 202 is self tapping, its advancement into the bone tissue without a guide wire is similarly improved. The blunt-ended rod 232 is securely fastened to the cannulated driver 204 by way of a similar locking mechanism to the locking mechanism 220 on the guide rod 206 and the cannulated driver 204 operated to advance the cannulated screw 202 across the joint space 226 and into the second bone 228, as illustrated in FIGS. 7 and 7A. Thus, the screw 202 goes through the facet joint. In this manner, the first bone 224 and the second bone 228 are fixed together by way of the cannulated screw 202, and this fixation is made more structurally sound as a result of the bioactive agent provided to the joint space 226 between the first bone 224 and the second bone 228. The cannulated driver 204 is then disengaged from the cannulated screw 202 and the cannulated driver 204 and blunt-ended rod 232, which are still fastened together, are collectively removed from the patient.

While reference was previously made to the fixation of the C1 and C2 vertebrae, it should be appreciated by those skilled in the art that that bone fixation device 200 can be used during the fixation any number of different bones or bone fragments together. For example, the bone fixation device 200 can be used to fix two different cervical vertebrae together, as well as different thoracic and lumbar vertebrae. In general, the bone fixation device 200 can be used to fix together two bones that share an articulated joint, or a bone and a bone fragment.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A bone fixation device kit, comprising:
a self tapping screw having an aperture extending along a longitudinal axis of the screw;
a driver having an aperture extending from a proximal end to a distal end along a longitudinal axis of the driver, the distal end being configured to engage the screw such that the aperture of the driver and the aperture of the screw are coaxially aligned, thereby defining a lumen;
a guide rod having a tip for piercing bone tissue, the guide rod being shaped and dimensioned to extend through the lumen and configured to fasten to the driver; and
a blunt-ended rod shaped and dimensioned to extend through the lumen.

2. The kit as recited in claim 1 in which the guide rod includes a locking mechanism for fastening the guide rod to the driver, and the tip of the guide rod is tapered.

3. The kit as recited in claim 2 in which the locking mechanism is a series of threads and the aperture extending through the driver includes a series of similarly configured threads so that the guide rod can be threaded into the aperture.

4. The kit as recited in claim 1 in which the blunt-ended rod is further configured to fasten to the driver.

5. The kit as recited in claim 4 in which the blunt-ended rod includes a locking mechanism for fastening the blunt-ended rod to the driver.

6. The kit as recited in claim 5 in which the locking mechanism is a series of threads and the aperture extending through the driver includes a series of similarly configured threads so that the blunt-ended rod can be threaded into the aperture.

7. The kit as recited in claim 1 in which the blunt-ended rod is for advancing a bioactive agent through the lumen, and the kit includes a bioactive agent selected from at least one of a bone morphogenetic protein and a demineralized bone matrix.

8. A method for fixing a first bone to a second bone, the method comprising:
a) providing:
a cannulated self tapping screw;
a cannulated driver configured to coaxially engage the cannulated screw;
a guide rod shaped and dimensioned to engage the cannulated screw and cannulated driver, and having an end for piercing a bone tissue;
a blunt-ended rod shaped and dimensioned to engage the cannulated screw and cannulated driver, and having a blunt end;
b) engaging the cannulated screw with the cannulated driver to form a lumen therethrough;
c) advancing the guide rod through the lumen;
d) fastening the guide rod to the cannulated driver;
e) operating the cannulated driver to collectively advance the cannulated screw and guide rod through the first bone until the guide rod pierces a surface of the second bone, forming a pilot hole therein;
f) removing the guide rod from the lumen;
g) fastening the blunt-ended rod to the cannulated driver;
h) operating the cannulated driver to collectively advance the cannulated screw and blunt-ended rod through the second bone, thereby fixing the first bone to the second bone; and
i) disengaging the cannulated driver and blunt-ended rod from the cannulated screw.

9. The method of claim 8 wherein step g) comprises providing a bioactive agent to the lumen, advancing the blunt-ended rod through the lumen so that the bioactive agent is dispersed out a distal tip of the screw into a space between the first bone and the second bone, and thereafter fastening the blunt-ended rod to the cannulated driver.

10. The method as recited in claim 9 in which the bioactive agent is formulated to aid a fixation of the first bone to the second bone.

11. The method as recited in claim 9 in which the bioactive agent is at least one of a bone morphogenetic protein and a demineralized bone matrix.

12. The method as recited in claim 9 in which the space in which the bioactive agent is dispersed is a joint space or a disc space.

13. The method as recited in claim 8 in which the guide rod includes a threaded portion and the end of the guide rod is tapered, the cannulated driver includes a similarly configured threaded portion, and the guide rod is fastened to the cannulated driver in step d) by threading the guide rod into the cannulated driver.

14. The method as recited in claim 8 in which the blunt-ended rod includes a threaded portion, the cannulated driver includes a similarly configured threaded portion, and the blunt-ended rod is fastened to the cannulated driver in step g) by threading the guide rod into the cannulated driver.

15. The method as recited in claim 8 in which the first bone is at least one of a cervical vertebra, a thoracic vertebra, and a lumbar vertebra, and the second bone is at least one of a cervical vertebra, a thoracic vertebra, and a lumbar vertebra.

16. The method as recited in claim 8 in which the first bone is a C1 vertebra and the second bone is a C2 vertebra.

17. The method as recited in claim 8 in which at least one of the first bone and the second bone is a bone fragment.

18. The method as recited in claim 8 in which the first bone is a first bone fragment in a long bone, and the second bone is a second bone fragment in the long bone.

19. The method as recited in claim 18 in which the long bone is a bone in an arm.

20. The method as recited in claim 18 in which the long bone is a bone in a leg.
